(19) United States
(12) Patent Application Publication
(10) Pub. No.: US 2020/0406053 A1
(43) Pub. Date: Dec. 31, 2020

(54) Sublingual Light Therapy Device

(71) Applicant: Photonx Holdings, Inc., Oldsmar, FL (US)

(72) Inventor: Scot Johnson, Tampa, FL (US)

(21) Appl. No.: 16/914,235

(22) Filed: Jun. 26, 2020

Related U.S. Application Data
(60) Provisional application No. 62/866,632, filed on Jun. 26, 2019.

(51) Int. Cl. A61N 5/06

(52) U.S. Cl. CPC A61N 5/0603; A61N 5/062; A61N 2005/063; A61N 2005/0632; A61N 2005/0606

(57) ABSTRACT

A sublingual light therapy device for use in photonic infusion therapy of sublingual tissues. The disclosed device is optimized to direct therapeutic light directly to the targeted tissues while providing comfort and convenience to the patient.

### FIELD OF THE DISCLOSURE

The present disclosure generally relates to devices and methods for processing light.

### BRIEF SUMMARY OF THE DISCLOSURE

The subject matter presented herein provides a sublingual light therapy device useful for a variety of applications. In one embodiment of the inventive concept, a sublingual light therapy device includes a light conduit connected to a mouthpiece, with one end of the light conduit connected to a light source and the other end disposed directly under the tongue of a patient.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The foregoing disclosure will be best understood and advantages thereof made most clearly apparent when consideration is given to the following detailed description in combination with the drawing figures presented. The detailed description makes reference to the following drawings:

FIG. 1 shows a sublingual light therapy device in use;

FIG. 2 shows the sublingual light therapy device shown in FIG. 1 from a side view;

FIG. 3 shows an exploded isometric view of the upper end of the light therapy device of FIG. 2;

FIG. 4 shows an exploded isometric view of the lower end of the light therapy device of FIG. 2;

FIG. 5A shows the mouthpiece suitable for use in the sublingual device, viewed from an oblique angle;

FIG. 5B shows the mouthpiece of FIG. 5A viewed from the top;

FIG. 5C shows the mouthpiece of FIG. 5A viewed from the left side;

FIG. 5D shows the mouthpiece of FIG. 5A viewed from the front;

FIG. 5E shows a section view along the central plane of the mouthpiece of FIG. 5A viewed from the right side;

FIG. 6A shows a left end view of a locking nut suitable for use with the present disclosure;

FIG. 6B shows a side view of the locking nut shown in FIG. 6A;

FIG. 6C shows a section view along the principal central longitudinal plane of the locking nut shown in FIGS. 6A and 6B;

FIG. 6D shows a right end view of the locking nut shown in FIGS. 6A-6C;

FIG. 7A shows a left end view of an output tip suitable for use with the present disclosure;

FIG. 7B shows a side view of the output tip shown in FIG. 7A;

FIG. 7C shows a right end view of the output tip shown in FIGS. 7A and 7B; and

FIG. 8 shows a side view of a protective sleeve suitable for use with the present disclosure.

### DETAILED DESCRIPTION OF THE DISCLOSURE

A device for light therapy is provided herein. The following detailed description provides certain specific embodiments of the subject matter disclosed. Although each embodiment represents a single combination of elements, the subject matter disclosed herein should be understood to include sub-combinations of the disclosed elements. Thus, if one embodiment comprises elements A, B, and C, and a second embodiment comprises elements B and D, then the inventive subject matter is also intended to include other remaining combinations of A, B, C, or D, even if not explicitly disclosed herein.

The present disclosure relates to a photonic infusion therapy end-effector optimized for the sublingual mucosa, providing improved quantum yield, accurate and consistent dosing, patient comfort and convenience.

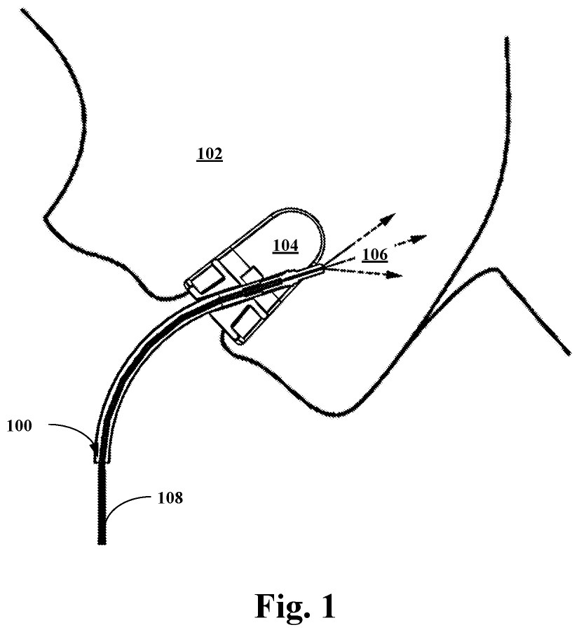

FIG. 1 shows a sublingual light therapy end-effector 100 in use within the mouth of photonic therapy patient 102. Mouthpiece 104 provides a photonic emission 106 radiating outward in a narrow emission cone. The photonic emission 106 of the sublingual light therapy end effector 100 is directed into the sublingual region underneath the patient's tongue. The angle of the cone of emission 106 is defined by the numerical aperture of optical fiber waveguide 108. Thus, the photonic emission aperture of mouthpiece 104 infuses a narrow emission of photonic energy 106, at least partially incident upon the sublingual space, which includes the ventral surface of the tongue.

In order to ensure that the therapy is applied properly and completely, sublingual light therapy end effector 100 is designed to enhance the comfort of patient 102 during treatment. Patient adherence and compliance with a treatment regimen is believed to be significantly affected by patient perception as to the clinical effectiveness the treatment as well as the convenience and relative comfort/discomfort involved in receiving said treatment.

If a patient perceives the treatment as not improving their health status within a desired period of time, that patient may never begin or may discontinue treatment prior to fully completing a prescribed treatment regimen. Similarly, if the patient perceives the treatment as too inconvenient to obtain, too uncomfortable to endure, or both, relative to the anticipated health benefits, the patient may discontinue treatment prior to fully completing a prescribed treatment regimen.

Sublingual end-effector 100 is designed to avoid limiting a patient's ability to breathe and communicate. Observation of these requirements mitigates potential phobic responses to placement of a sublingual photonic therapy end-effector 100 in a patient's mouth for any appreciable time, avoids safety risks associated with patients' inability to communicate, and mitigates the severity associated with muscles and joint spaces of the jaw.

Sublingual photonic therapy end-effector 100 is designed to function comfortably and direct emission 106 properly across a range of patient head motions and positions without restraint or discomfort. The optimal angles of the geometry may vary by application, as described in further detail below.

Sublingual end effector 100 may also be designed to facilitate the ability of patient 102 to multitask during the treatment session. This may include, for example, the ability to work while seated at a desk during sublingual photonic therapy. Other activities that may be conducted while seated upright include but are not limited to using a smartphone, watching television, having a conversation, and reading a book. A similar alignment is also anticipated during activities conducted while the patient is generally vertically oriented.

In one preferred embodiment of the present disclosure, photonic waveguide 108 extending outward from mouthpiece 104 is an optical fiber. Optical fiber is typically comprised of multiple concentrically-oriented layers of cylindrically-shaped extruded materials. Depending on the materials incorporated, the final optical fiber can maintain stated optical specifications up to a minimum bend radius, beyond which optical specifications begin to degrade. A minimum bend radius is given as the range between short term and long term minimum bend radii. The long term minimum bend radius may be maintained indefinitely and maintain stated optical specifications. The short term minimum bend radius can only be maintained for short durations, beyond which time attenuated optical performance becomes irreversible.

The design of sublingual end-effector 100 incorporates a structure which will allow no less than a bend radius equal to the long-term minimum bend radius. Any advantage conveyed by a smaller bend radius must be weighed against a loss of optical transmission performance. In sublingual photonic infusion therapy, it may desirable to issue multiple wavelengths of photonic energy simultaneously. Transmission of multiple non-coherent photonic wavelengths may be accommodated using multimode optical fiber. The diameter of the core, or central portion of the optical fiber and carrying the majority of photonic energy, is generally directly related to how much photonic energy an optical fiber can transmit in this embodiment. As such, a larger diameter optical fiber core will allow more photonic energy to infuse into the sublingual region and ventral surface of the tongue, and reduces the time required to infuse the photonic energy dosage.

A variety of different optical fibers may be employed in various embodiments of the present disclosure. These may include, as examples: i) ThorLabs FT400UMT Ø400 um Core Multimode Optical Fiber with a numerical aperture 0.39 NA and high hydroxyl content (High OH); ii) ThorLabs FT600UMT Ø600 um Core Multimode Optical Fiber with a numerical aperture 0.39 NA and high hydroxyl content (High OH); iii) ThorLabs FT800UMT Ø800 um Core Multimode Optical Fiber with a numerical aperture 0.39 NA and high hydroxyl content (High OH); iv) ThorLabs FT400EMT Ø400 um Core Multimode Optical Fiber with a numerical aperture 0.39 NA and low hydroxyl content (Low OH); v) ThorLabs FT600EMT Ø600 um Core Multimode Optical Fiber with a numerical aperture 0.39 NA and low hydroxyl content (Low OH); and vi) ThorLabs FT800EMT Ø800 um Core Multimode Optical Fiber with a numerical aperture 0.39 NA and low hydroxyl content (Low OH).

The long-term minimum bend radii optical fibers of the above-listed optical fibers are 40 mm, 60 mm, and 80 mm respectively. A numerical aperture of 0.39 is employed in one embodiment of the present disclosure, allowing a larger diameter cone of light to both enter and exit the optical fiber than in other commonly obtained optical fiber numerical apertures (e.g. 0.22 NA). Where the designer desires transmission of photonic energy of wavelengths less than about 400 nm, high hydroxyl content (High OH) fiber is advantageous.

Fibers contemplated here account for additional considerations as relate to biocompatibility, sterilization and florescence spectroscopy. In one embodiment, FT600UMT is used for the optical fiber waveguide, comprised of a 600 μm core, a 630 μm cladding, and a 1040 μm outer coating. It is anticipated the outer coating of the waveguide may come into contact with patient skin and bodily fluids. The outer coating of this optical fiber is made of Tefzel™, a plastic commonly utilized in medical devices requiring ISO 10993 conformity for skin and bodily fluid contact. This optical fiber is rated by the manufacturer for high-energy radiation resistance, a requirement for gamma sterilization processing, as well as continuous exposure at 150° C. for 20,000 hours, a requirement for autoclave processing.

FT600UMT is made of silica with a High OH, preferable for transmission of lower wavelengths below about 700 nm and necessary for any appreciable transmission below about 400 nm. Transmission of wavelengths below 400 nm, and within the UVA1 spectral band extending to 350 nm, allows for photoexcitation of endogenous photophilic structures known as fluorophores. Fluorophores release (i.e. re-emit) a portion of the photonic energy absorbed as a photon at a higher wavelength than that of the absorbed photon (i.e. at a lower energy than that of absorbed photon) which may be visible to the naked eye (as UV wavelengths reside below the visible range of photons), a phenomena called ‘fluorescence’. An image capture device, positioned such that the fluorescence can be captured and visualized, can use this fluorescence data to see the structures which contain the photo-excited ‘fluorescing’ fluorophores. In a preferred embodiment, a secondary optical waveguide with a distal aperture adjacent to a photonic emission aperture, emitting photonic energy containing UVA1 wavelengths, is used to collect fluorescence data and deliver that to a camera. Such an application may be useful, for example, in detection of certain cancers.

The radius of the arc of waveguide 108 is preferably equivalent to or greater than the long-term minimum bend radius for the optical fiber employed. The upper portion of waveguide 108 should ideally be restricted to a bend radius of at least the long-term minimum bend radius for the fiber used. In certain embodiments, this may be accomplished by the use of a protective sleeve.

As will be shown, the apparatus and method taught herein is advantageous in combination with a novel armature providing consistent sublingual placement of the photonic emission aperture of the mouthpiece 104 relative to the sublingual space of the patient 102. The present disclosure, in some embodiments, is described as a sublingual end-effector ‘system,’ which includes an armature or support, easily and comfortably held within the patient mouth, providing consistent sublingual placement of the photonic emission aperture relative to the sublingual space and which minimally impedes airflow and communication.

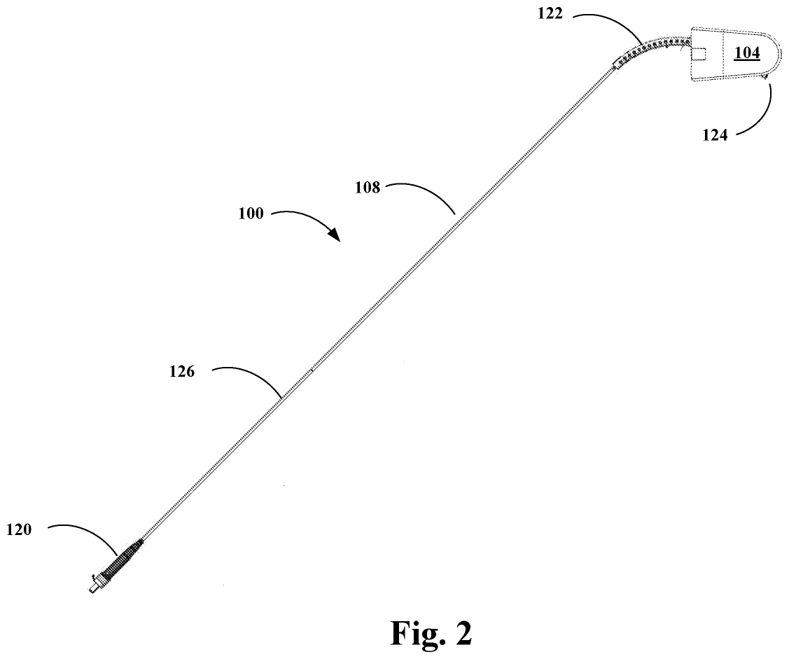

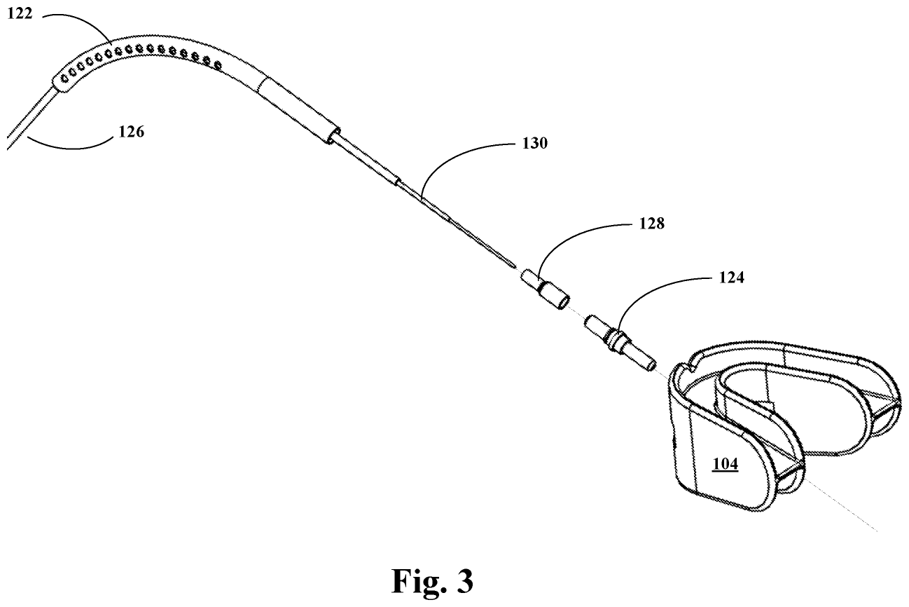

Turning now to FIGS. 2 and 3, FIG. 2 shows sublingual end-effector 100 from a side view. As seen in these figures, sublingual end-effector 100 includes an optical fiber waveguide 108 terminated at the proximal end by waveguide input connector 120. Waveguide input connector 120, in one embodiment of the immediate application, is an SMA-905 connector, such as ThorLabs part #10640A, designed with an internal bore diameter of 640 μm suitable for insertion of an optical fiber waveguide after the coating is stripped away. The waveguide input connector 120 may include the ferrule, knurled locking nut, and C-clip from ThorLabs part #1064A as well as the crimp sleeve and strain relief boot from ThorLabs part #190088CP.

A protective sheath 122 is located at the distal end of the end-effector, adjacent to waveguide output tip 124. Protective sheath 122 may be a precision-bent, laser cut (and drilled) 6TW Gauge passivated 304-series stainless steel hypodermic tube. Waveguide 130 is bent and held at no less than the long-term minimum bend radius for the optical fiber type.

In one embodiment, the entire end-to-end center length of the sublingual end-effector 100 is 25.5 inches±0.5 inches in an embodiment of the present disclosure. In the inventor's own experiences have reliably shown a design-for-manufacturing (DFM) overall assembly length tolerance of ±0.5 inches is more than reasonable in terms of limiting production yield due to out-of-tolerance length assemblies.

Furcation tube 126 protects optical fiber 130. In one embodiment, furcation tube 126 is a length of ThorLabs part #FT020 orange reinforced 2.0 mm furcation tubing. The optical fiber 130 passes through furcation tube 126 concentrically.

As seen in FIG. 3, at the distal end of the sublingual end-effector 100, waveguide output tip 124 is shown, as well as a length of expanded heat shrink tubing 128. In one embodiment, waveguide output tip 124 is the ferrule from ThorLabs part #1064A. In another embodiment, expanded heat shrink tubing 128 is FEP 1.6:1 Fractional Size 3/16 inch heatshrink tubing supplied by Zeus™. Heat shrink tubing 128 has a working temperature of up to 200° C., substantial radiation resistance, and satisfies ISO 10993 requirements.

In consideration of sterilization compatibility with autoclaving, in a preferred embodiment of the present disclosure, waveguide input connector 120 may be modified as comprising a ThorLabs part #10640V high-temperature rated (up to 250° C.) 304-series stainless steel SMA-905 connector assembly and a high-temperature rated (up to 250° C.) 304-series stainless steel sleeve such as ThorLabs part #FTSB1 modified as appropriate. Furcation tubing 126 would not be included due to incompatibility with autoclave temperatures, leaving the coating of the optical fiber to protect the cladding and core therein. Waveguide output tip 124 may be the ferrule from ThorLabs part #10640V high-temperature rated (up to 250° C.) 304-series stainless steel SMA-905 connector. Finally, it should be noted that the epoxy used in the construction of the assembly should be compatible with high-temperatures. In one preferred embodiment, the epoxy used is ThorLabs part #353NDPK high-temperature rated (up to 250° C.) color-changing 2-part epoxy.

In consideration of MRI compatibility, in a preferred embodiment of the present disclosure, all metallic components can be made of nonmagnetic metals (e.g. Titanium) or of other non-metallic and non-ferromagnetic materials, including ceramics and plastics which can be injection molded and machined.

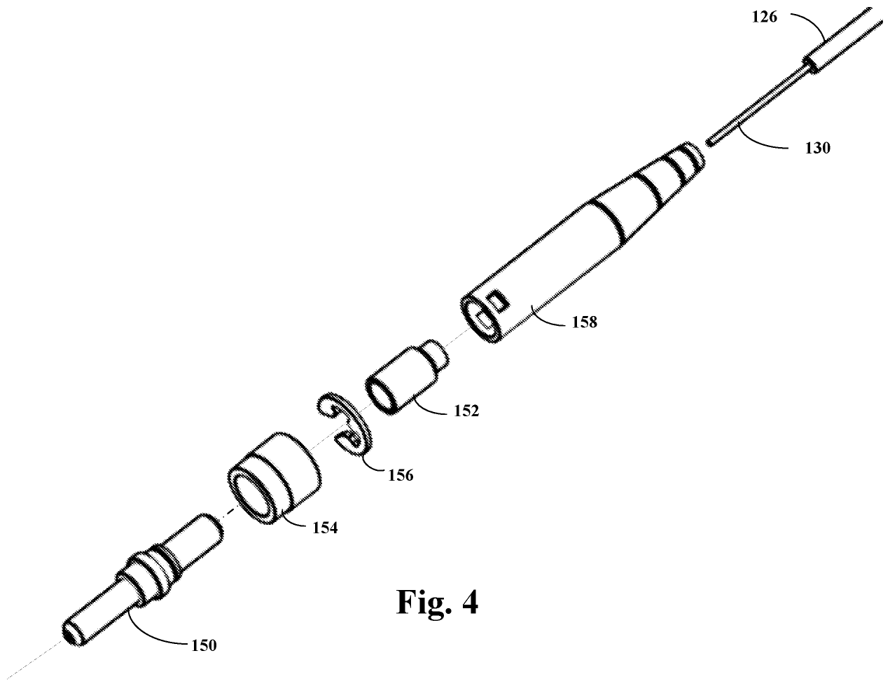

Turning now to FIG. 4, waveguide output tip 150, autoclave-compatible furcation tube 126 with optical fiber waveguide 130 running therethrough, and a cut-to-length expanded section of heatshrink tubing 152 are shown prior to assembly. Optical fiber waveguide 130 has been stripped and cut-to-length for attachment to/within the waveguide output tip 150. Furcation tubing 126 has been cut-to-length, and any structural fibers (e.g. Kevlar®) contained within have been cut, organized and arranged for attachment to waveguide output tip 150. Optical fiber-compatible epoxy may be used as a fixative. The proximal ends of furcation tube 126 and optical fiber waveguide 130 have been cut-to-length, however as will be described cannot be assembled for connectorization and polishing at this stage.

It is important to ensure the post-shrinking outer diameter of heatshrink tubing 152 is no greater than 0.180 inches at any point, and that the leading-edge of the shrunken heatshrink tubing 152 is incident to, and does not go beyond, the face of the waveguide output tip 150.

Any adhesives have been allowed to cure under the appropriate conditions and are cured. This distal end subassembly proceeds to polishing on an automated polishing machine equipped with standard SMA-905 tooling. In embodiments of the immediate teachings, the face of optical fiber waveguide 130 is polished and inspected to 0.02 μm, 0.3 μm, or 1 μm.

Following polishing and inspection, the assembly proceeds with the removal of the knurled locking nut 154 and C-clip 156. Knurled locking nut 154 is removed by sliding it backwards and off of the cut ends of furcation tubing 126 and optical fiber 130 at the proximal end of the assembly. After removal of knurled locking nut 154 and C-clip 156 from the waveguide output tip 150, the distal end of the end-effector including polished waveguide output tip, the shrunken heatshrink tubing 152, furcation tubing 126 and optical fiber 130 forms a completed assembly. In the embodiment shown in FIG. 4, a strain relief boot 158 is disposed over the assembled connector.

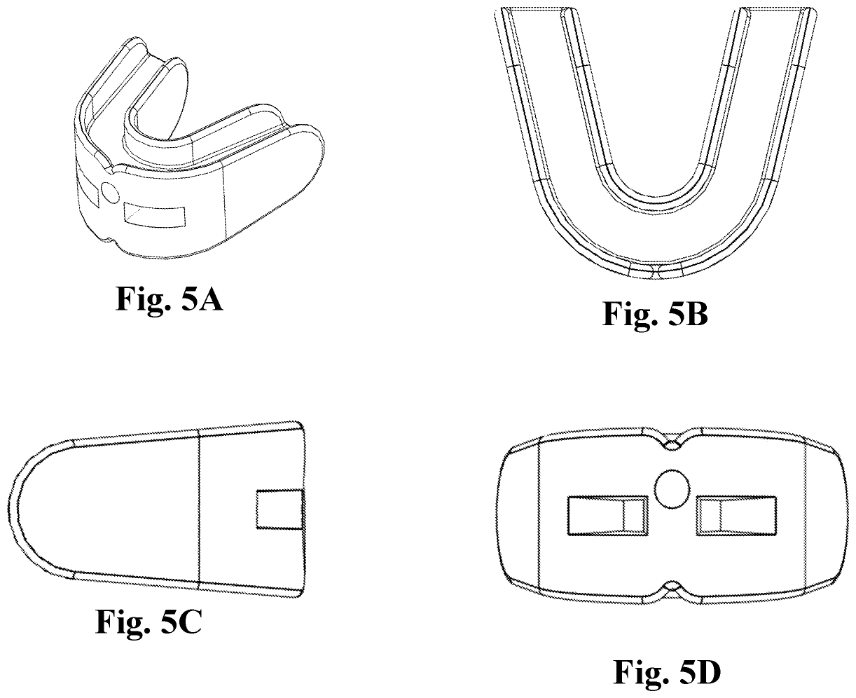

FIGS. 5A-5D show mouthpiece 104 viewed from an oblique angle, top, left side, front and right side, respectively. FIG. 5E shows a section view along the central plane of the mouthpiece of FIG. 5A viewed from the right side.

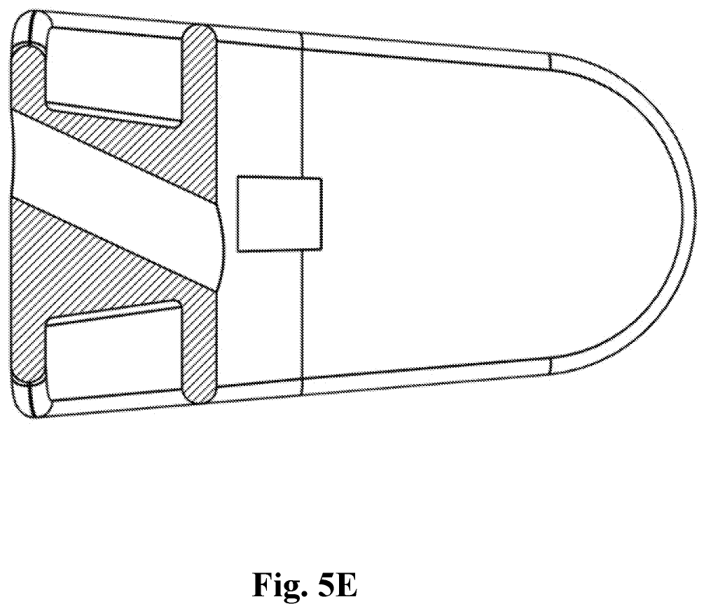

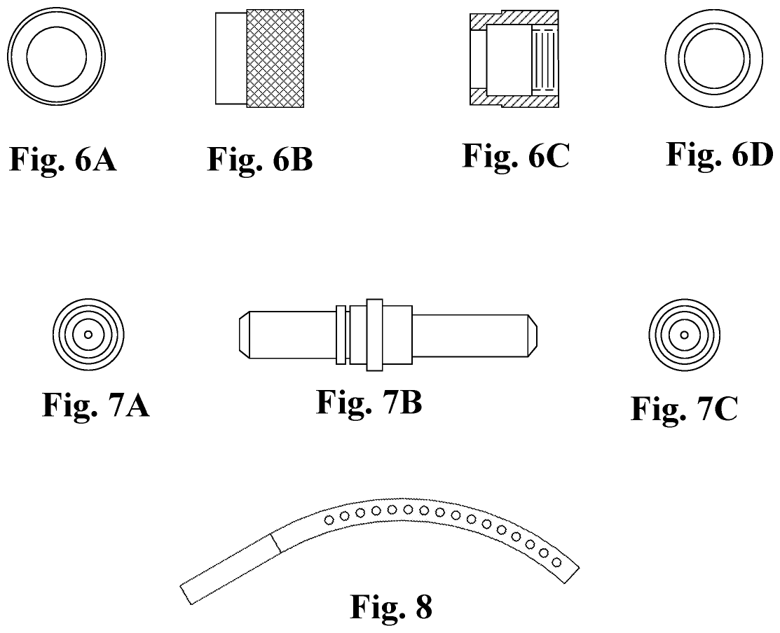

FIGS. 6A, 6B and 6D show locking nut 154 viewed from the left end, side and right end, respectively. FIG. 6C shows a section view along the principal central longitudinal plane of locking nut 154.

FIGS. 7A-7C show a ferrule suitable for use as an optical output within the present disclosure, viewed from the left end, side and right end view, respectively.

FIG. 8 shows a side view of a protective sleeve suitable for use with the present disclosure.

The present disclosure has been presented in connection with certain exemplary embodiments. It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A sublingual end-effector comprising:
an optical fiber waveguide, having a waveguide input connector at a proximal end thereof and an output tip at a distal end thereof; and
a mouthpiece, secured to the output tip of the optical fiber waveguide.

2. The sublingual end-effector of claim 1, further comprising:
a protective sheath, disposed around the optical fiber waveguide, adjacent to waveguide output tip.

3. The sublingual end-effector of claim 2, wherein the protective sheath comprises a rigid, curved tube having a fixed bend radius.

4. The sublingual end-effector of claim 3, wherein the protective sheath has a bend radius of at least 40 mm.

5. The sublingual end-effector of claim 1, wherein the output tip of the optical fiber waveguide is disposed within a downwardly-angled aperture within the mouthpiece to direct photonic emissions from the output tip of the optical fiber waveguide into the sublingual region of a patient.
